(19) United States
(12) Patent Application Publication
(10) Pub. No.: US 2011/0190621 A1
(43) Pub. Date: Aug. 4, 2011

(54) Methods and Systems for Regional Synchronous Neural Interactions Analysis

(76) Inventors: Todd A. Verdoorn, Minneapolis, MN (US); Michael Cassano, Minneapolis, MN (US); Rajasimhan Rajagovindan, St. Louis Park, MN (US); Steven Lebow, Minneapolis, MN (US)

(21) Appl. No.: 13/019,135

(22) Filed: Feb. 1, 2011

Related U.S. Application Data
(60) Provisional application No. 61/337,201, filed on Feb. 1, 2010.

(51) Int. Cl. A61B 5/055

(52) U.S. Cl. 600/411

(57) ABSTRACT

Systems and methods for quantifying neurophysiologic activity of a subject. A set of subject data representing a time series of neurophysiologic activity acquired by each of a multiplicity of spatially distributed sensors arranged to detect neural signaling in the subject is received. A time series of data obtained from each of the sensors is associated with a corresponding neural population within the brain of the subject. Interaction sets among at least two neural populations in the brain of the subject are determined based on a statistical analysis of a plurality of time series of data from a corresponding plurality of sensors. A plurality of regional groupings of neural populations is stored, with each one of the plurality of regional groupings encompassing a plurality of neural populations having a predefined relationship. An aggregated representation of cross-regional interactions between the neural populations across a selected plurality of the regional groupings is produced based on a selected subset of the interaction sets.

### PRIOR APPLICATION

This application claims the benefit of U.S. Provisional Application No. 61,337,201, filed Feb. 1, 2010, which is incorporated by reference herein in its entirety.

### FIELD OF THE INVENTION

This invention relates generally to healthcare and, more particularly, to automated analysis of neurophysiologic data.

### BACKGROUND OF THE INVENTION

Aspects of the invention are directed to improvements in the analysis of synchronous neural interactions measurements that are disclosed in U.S. Pub. No. 2008/0091118, entitled “ANALYSIS OF BRAIN PATTERNS USING TEMPORAL MEASURES,” which is incorporated by reference herein. U.S. Pub. No. 2008/0091118 discloses a temporal approach to analyzing interactions within the brain and subsequently producing a dynamic model, which represents patterns of neural activity. The dynamic model can be further analyzed to evaluate the presence, if any, of various disease states or other conditions, such as the effects of psychoactive drugs.

One continually present challenge in this area is in ensuring accurate, reliable, and repeatable performance in generating the dynamic brain model and in evaluating the brain model against the disease states or other conditions. Therefore, a need exists for a system that provides robust, quantitative empirical data representing a patient's brain function and the health thereof, and to do so in a simple, quick, and computationally efficient manner.

### SUMMARY OF THE INVENTION

Aspects of the invention are directed to quantifying neurophysiologic activity of a subject. The invention may be carried out in a computer system, having computer hardware, including a data processor and a data input. A set of subject data representing a time series of neurophysiologic activity acquired by each of a multiplicity of spatially distributed sensors arranged to detect neural signaling in the subject is received via the data input. A time series of data obtained from each of the sensors is associated with a corresponding neural population within the brain of the subject. The association may be performed as part of the processing by the data processor, or it may be inherent in the received set of subject data. Interaction sets among at least two neural populations in the brain of the subject are determined based on a statistical analysis of a plurality of time series of data from a corresponding plurality of sensors. The interaction sets may be interactions among pairs of neural populations, for instance, as measured by a corresponding pair of spatially-distributed sensors. A plurality of regional groupings of neural populations is stored, with each one of the plurality of regional groupings encompassing a plurality of neural populations having a predefined relationship. The groupings may be based on any number of predefined relationships, such as general location within the brain, belonging to a common brain structure, belonging to a common functional center of the brain, or having interaction sets that have a similar distance between their respective neural populations. An aggregated representation of cross-regional interactions between the neural populations across a selected plurality of the regional groupings is produced based on a selected subset of the interaction sets.

Aspects of the invention further include program instructions for carrying out the data processing described above, as well as a method for transmitting the subject data for remote processing, and receiving the result of that remote processing.

There are a number of advantages of the regional approach including, for instance, reduction of data dimensionality, decreased variability between subjects and instruments, and an ability to implement empirical testing of predefined hypotheses regarding regional changes in brain function. A number of other advantages will become apparent from the following Detailed Description of the Preferred Embodiments.

### BRIEF DESCRIPTION OF THE DRAWINGS

The drawings illustrate generally, by way of example, but not by way of limitation, various embodiments discussed in the present document.

FIG. 1 illustrates an exemplary MEG instrument.

FIGS. 2A and 2B illustrate synchronous dynamic networks from a subject in a as a visual representation.

FIG. 3 illustrates an exemplary classification plot produced utilizing canonical discriminant functions.

FIGS. 4A-4C illustrate spatial patterns for three separate sensors.

FIGS. 5A-5C illustrate spatial patterns for three more sensors.

FIGS. 6-9B illustrate various examples of massively interconnected networks.

FIG. 10 illustrates another exemplary classification plot produced utilizing canonical discriminant functions.

FIG. 11 illustrates a network implemented example of the present subject matter.

FIGS. 12A and 12B illustrate methods corresponding to examples of analyzing a subject according to various aspects of the invention.

FIG. 13 is a diagram illustrating an overview of a process of analyzing a subject according to one aspect of the invention.

FIG. 14 is an information flow diagram illustrating exchange of information in a system according to one aspect of the invention.

FIG. 15 illustrates an example set of brain regions as defined according to one embodiment, and depicts groupings of MEG sensors situated over those regions.

FIG. 16 illustrates an example set of structure-based brain regions as defined according to one embodiment.

FIG. 17 illustrates an example set of distance-based sensor pairings according to one embodiment.

FIG. 18 illustrates Alzheimer's disease-induced changes in connectivity depending on sensor distance.

FIG. 19 illustrates the magnitude and direction of instantaneous or zero-lag correlation values vary regularly with the distance between sensors.

FIGS. 20A-20F depict representative source code for defining regions and calculating regional values, according to one embodiment.

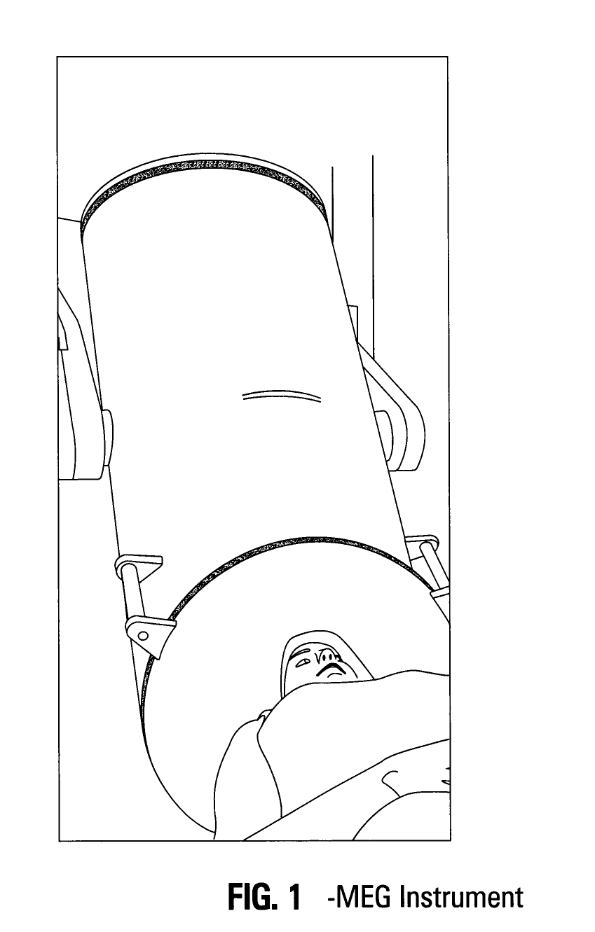

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims and their equivalents.

### DETAILED DESCRIPTION OF THE PREFERRED EMBODIMENTS

Aspects of the invention are directed to synchronous neural interactions (SNI) by which to assess dynamic brain function. Dynamic brain function refers to brain function that is observable at high temporal resolution using a measurement of the electrical activity within the brain. Neuronal activity in the brain produces both a magnetic signal and an electrical signal. A magnetic signal corresponding to the brain can be detected using a magnetoencephalography (MEG) sensor and an electrical signal can be detected using an electroencephalography (EEG) sensor. As used herein, an electromagnetic sensor can be used to detect either an electrical signal or a magnetic signal. Such measures may be taken using techniques such as MEG, EEG, or by a combination thereof. Other measurement techniques, either presently existing, or to be developed in the future, may be employed to generate a dynamic model of the electrical activity of the brain. For the sake of brevity, the example embodiments described below are based on data obtained via MEG measurements; however, it should be understood that principles of the invention may be adapted for data obtained using other measurement modalities. A modeling technique according embodiments of the invention includes measurement of dynamic synchronous interactions among neuronal populations which correspond to brain function.

For example, in addition to MEG and EEG sensors, other modalities can be used to collect temporal data from the brain. For example, a functional magnetic resonance imaging (fMRI) is a modality that provides data corresponding to the behavior of electron spins within the body during a particular activity. fMRI can detect the uptake of oxygen by neurons, which is an indicator of those cells' activity. Functional positron emission tomography (fPET) is another modality that detects gamma ray radiation emitted from radioactive substances introduced into the body. fPET can detect the uptake of glucose by neurons as an indicator of cell activity. Data from such modalities can be used in various embodiments to generate by the subject data to be input into the analytical system described herein.

Embodiments of the invention can be used for providing accurate, differential classification from among a variety of conditions. Some examples of such conditions include, but are not limited to, one or more of the following conditions: a normal condition, Alzheimer's Disease, pre-dementia syndrome, mild cognitive impairment, schizophrenia, Sjogren's Syndrome, post-traumatic stress disorder, alcoholism, alcohol impairment, fetal alcohol syndrome, multiple sclerosis, Parkinson's Disease, bipolar disorder, traumatic brain injury, depression, autoimmune disorder, a neurodegenerative disorder, pain, a disease affecting the central nervous system, or any combination thereof.

One method facilitates demarcation of ranges for healthy subjects, classifications for disease groups, measures of severity or degree with which a certain condition is manifested in a subject, and allows monitoring of changes in brain function coincident with disease progression or therapy intervention. In one example, the method can be used routinely for assessing dynamic brain function and aids in differential diagnosis and monitoring the effects of intervention. Classification scores and posterior probabilities can be obtained by which to quantify the severity of brain dysfunction and monitor its course, and the effect of treatment.

The exemplary method includes analysis of monitored data by way of dynamic, synchronous interactions among neuronal populations. The method can be used to discriminate between various brain impairments, including but not limited to subjects with AD, chronic alcoholism, MCI, multiple sclerosis, schizophrenia, PTSD, and Sjogren's syndrome, to name a few. The present subject matter can be used as a test for assessing dynamic brain function and serve as an aid in differential diagnosis. The present subject matter is also useful in drug development applications, including during all phases of the drug development cycle.

In one example embodiment of the present invention, time series analysis methods are applied to the MEG signal to estimate dynamic, moment-to-moment interactions between neuronal populations to predict motor behavior and music to derive synchronous neural networks involved in a task, and to assess their alteration in AD and chronic alcoholism. This time series analysis approach has proven very useful and promising for evaluating the status of brain function.

In one example embodiment, time series analyses is used to derive synchronous dynamic networks from single trials, unaveraged and unsmoothed, recorded simultaneously from 248 MEG sensors at 1 ms temporal resolution during an eye fixation task using the cross correlation function (CCF). For ease of illustration, much of the following description refers to interaction sets of two sensors, i.e., pairs of sensors. However, interaction sets of 3, 4, or more sensors can also be utilized in various embodiments. Also, it should be noted that, while a temporal resolution of +/−1 ms was found to provide particular benefits, other temporal resolutions may be employed, such as, for instance resolutions of +/−2 ms, +/−5 ms, +/−10 ms, and +/−25 ms in various applications.

This analysis yields visualizations of synchronous dynamic brain networks that are very similar and robust across healthy subjects. Note that certain advantages have been found using data obtained during an eyes-open idle state; however, other applications may be suitable for data obtained during eyes-closed patient states, idle or otherwise. The high density spatial sampling, the dynamic nature of the networks uncovered, and the robustness indicate utility as a test for assessing dynamic brain function at rest.

Statistically significant differences are noted in these networks between healthy subjects and subjects with AD, or MCI, as well as in chronic alcoholics during detoxification. In addition, CCF estimates of regional connectivities exhibit discriminatory power sufficient to classify individual subjects to particular groups (e.g. healthy, AD, MCI). Furthermore, genetic searching algorithms allow extension of this classification system to additional groups, including schizophrenia and chronic alcoholism. Statistically significant differences are also noted between healthy subjects and patients taking psychoactive drugs.

Genetic searching algorithms, or genetic algorithms (GA), have been developed to find genes in large arrays of chromosomes. A genetic algorithm is used in some embodiments of the present invention to search the large set of synchronous interactions among interaction sets (e.g., pairs) of neural populations in the brain (measured by a corresponding pair of sensors—e.g., 30,628 pairs when using 248 sensors—to find subset of these synchronous interactions that are able to predict the classifications of brain diseases and conditions.

Data preprocessing. Cardiac artifacts may be removed using an event-synchronous subtraction method. Due to the very short duration of the eye-fixation period (1 min), artifacts from eye blinks may be avoided altogether, but if present are detected and removed or blanked from the data.

Time series modeling of the MEG data. Analysis of single-trial, unaveraged data, following removal of the cardiac and/or eye blink artifacts benefits from the high temporal resolution and dynamic variation inherent in the MEG signal in order to assess functional interactions among large neural populations in a given task by calculating all cross correlation functions between all interaction sets (e.g., pairs) of the 248 sensors after prewhitening, i.e. converting the MEG time series to stationary, white noise series. This is achieved by modeling the raw series using AutoRegressive Integrative Moving Average (ARIMA) analysis, and taking the residuals. The CCF is then calculated for all possible interaction sets (e.g., pairs) of these “prewhitened” series at zero lag and various temporal lags. These CCFs can be regarded as connectivity weights in a massively interconnected neural network, where the 248 sensors serve as nodes. Synchronous dynamic networks are constructed using the partial correlations derived from the zero-lag and lagged CCFs. Zero-lag partial correlation between sensor I and sensor J is referred to as PCC_{IJ}^{O} and can take positive or negative values. An example of positive and negative interactions is shown in FIGa. 2A and 2B. PCC_{IJ}^{K} refers to lag-K partial correlation between sensor I and sensor J. K can take on positive and negative values, with negative lag reflecting time series in sensor I leading time series in sensor J, and positive lag reflecting time series in sensor I lagging time series in sensor J. Such lagged correlation and partial correlation is used to infer direction of causal influence. Approaches and applications using PCC_{IJ}^{O} are highlighted in the following examples; however the same applies to PCC_{IJ}^{O} without loss of generality and thus is not limited to PCC_{IJ}^{O}.

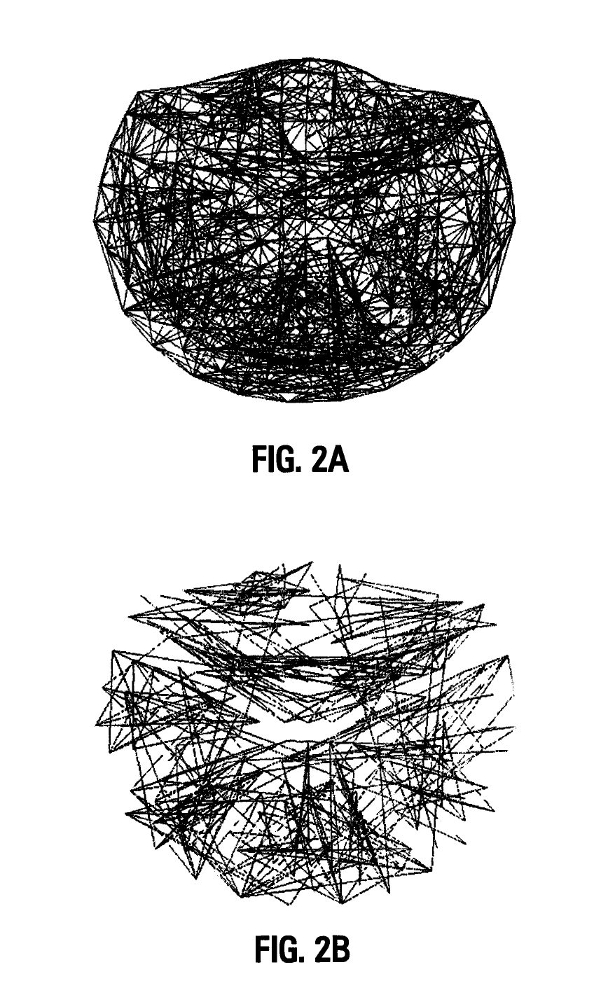

In FIGS. 2A and 2B, lines denote thresholded partial correlations (Fisher z-transformed). FIG. 2A illustrates positive partial correlations while and FIG. 2B illustrates negative partial correlations. There are 30,628 lines drawn (i.e. all possible pairs of 248 sensors) but only those exceeding the threshold below are depicted. The statistical significance threshold was adjusted to account for 30628 multiple comparisons according to the Bonferroni inequality: the nominal significance threshold is p<0.001, corresponding to an actual threshold used of p<0.001/30628 (=p<0.00000003). Analysis may be based on a 45-s long time period without averaging or smoothing.

Analysis is conducted to derive discriminant classification functions for certain groups of subjects with respect to selected measurements and then apply them to new cases to classify them in one of the original groups. This analysis yields posterior probabilities for classification to each group as well as a specific measure (e.g., squared Mahalanobis distance) which is the distance of the particular case from each of the classifying groups. This measure can serve to monitor potential changes in brain function to approximate that of different groups.

For example, consider AD. Data from subjects diagnosed with the disease (AD group) and subjects who are matched healthy controls (C) are used to derive two linear discriminant classification functions from the SNI data, one for AD and the other for C.

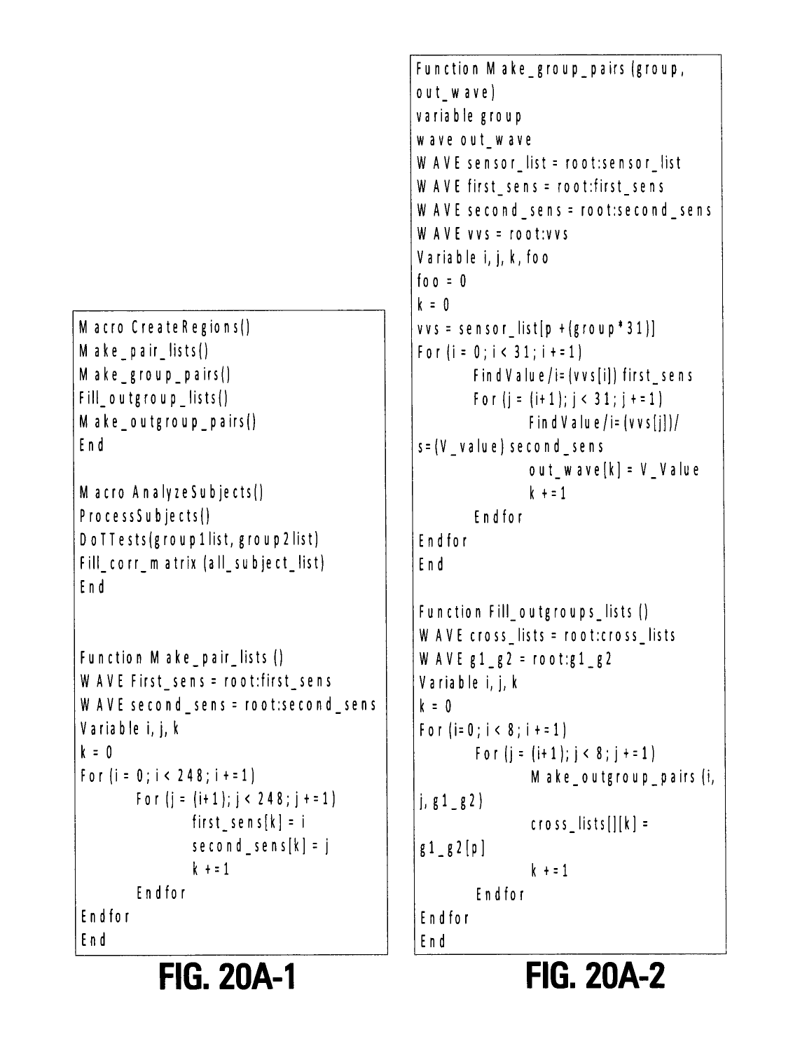

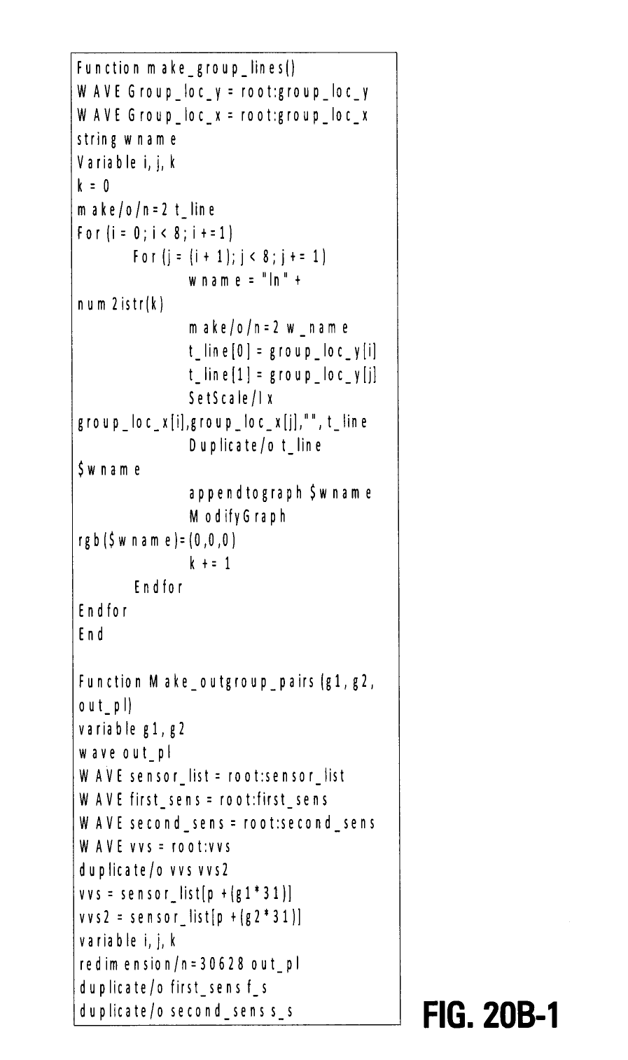

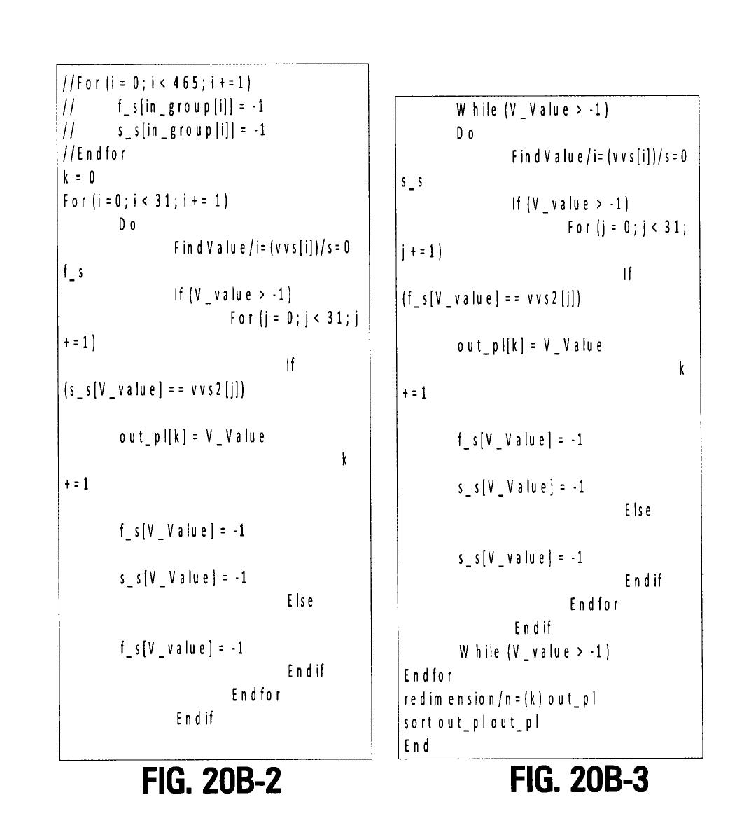

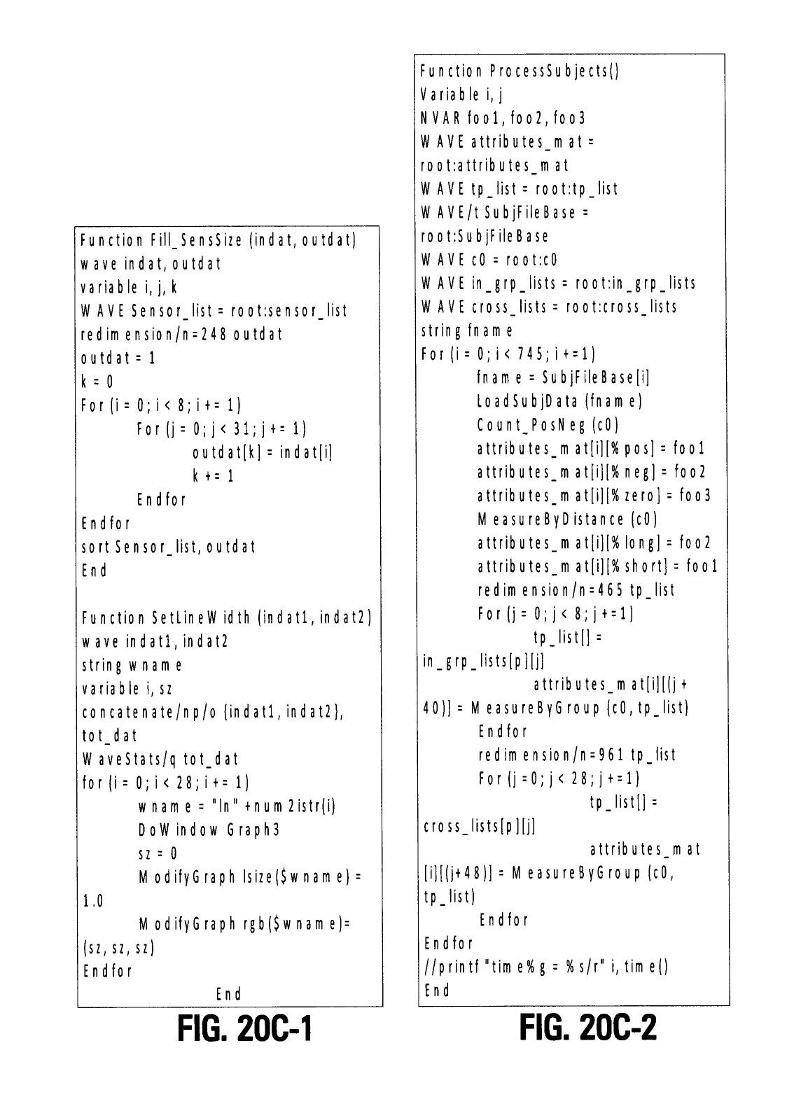

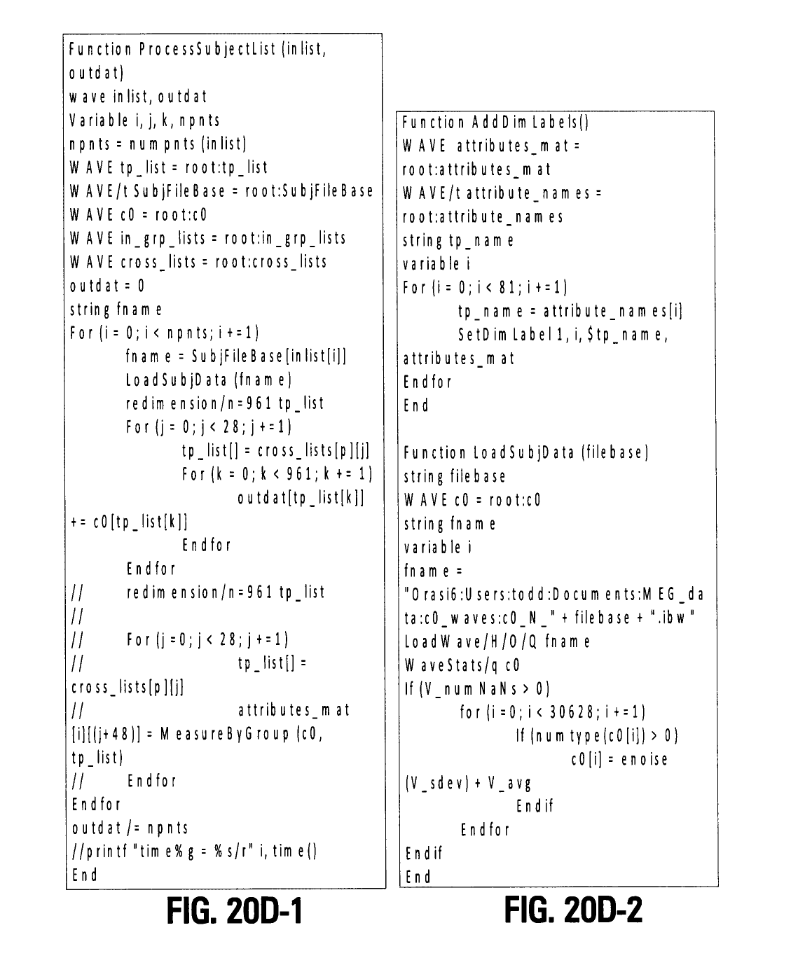

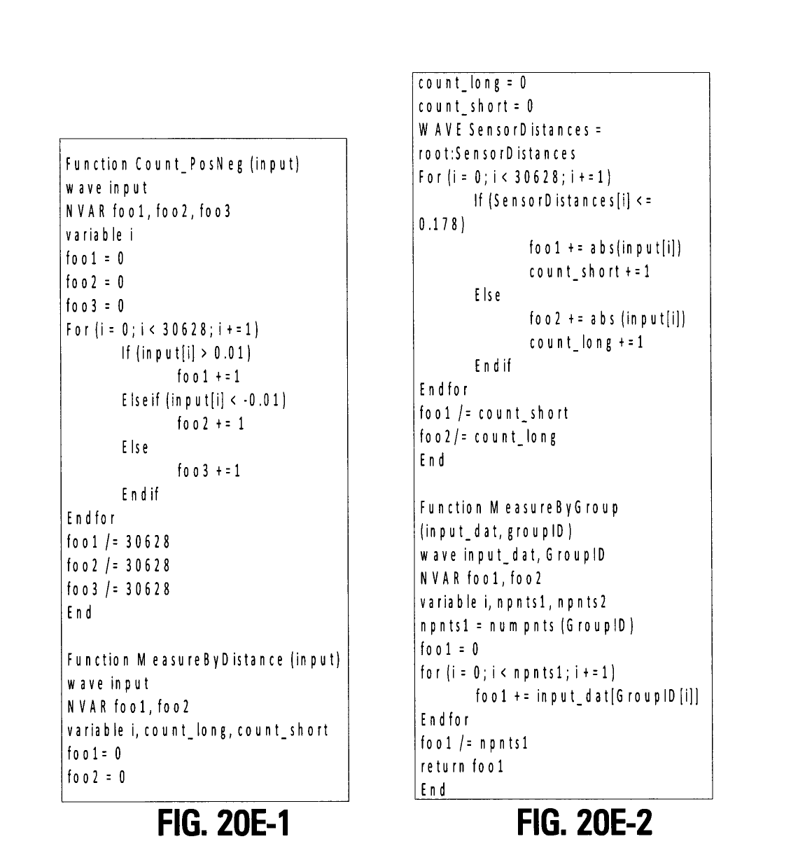

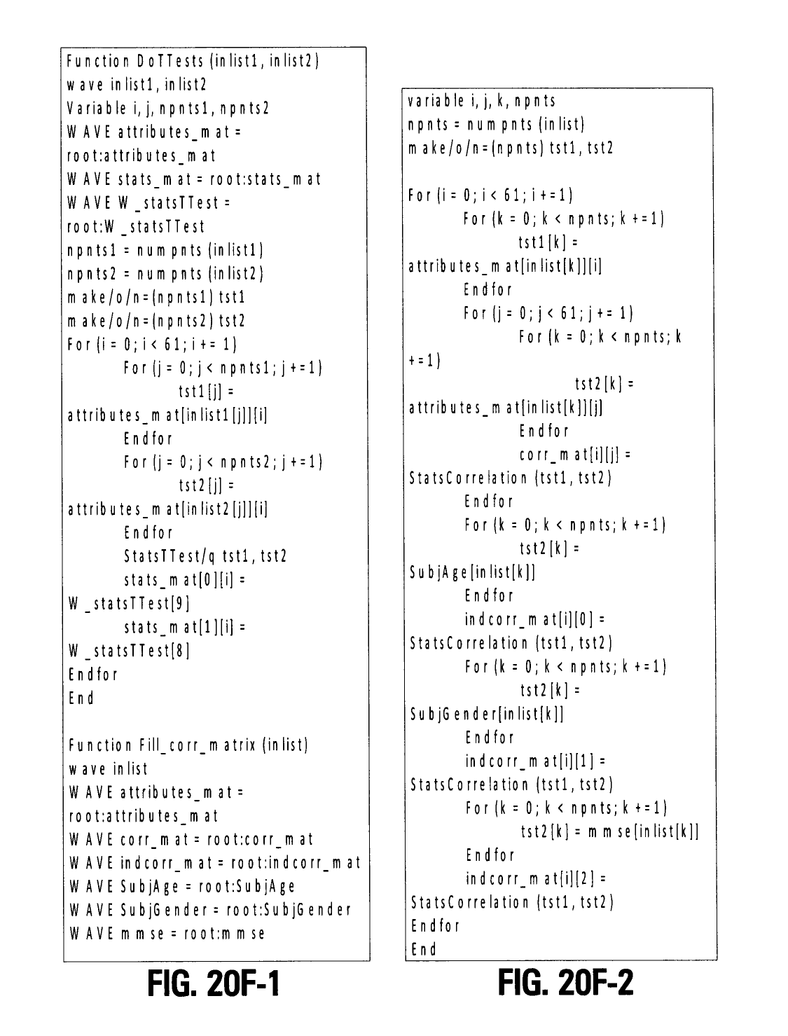

A new subject with a potential initial diagnosis of mild cognitive impairment (MCI) is subjected to a SNI test. By applying the AD and C classification functions, an estimate is made as to what extent the new subject is healthy or AD. This assessment is not binary but rather continuous, as measured by the squared Mahalanobis distances to the centers of the AD and C groups in the canonical discriminant functions plot. These relative distances can serve as monitors of disease progression (subject will become more “AD-like”), regression (more “C-like”), or effect of intervention (more “C-like” after drug treatment). Proximity refers to the relative distance and need not be displayed in graphical form in order to have meaning. Multivariate statistical analysis, such as linear discriminant classification analysis, can be used on a variety of conditions or diseases, including but not limited to, for example, AD, C, and MCI data, as well as subjects with Sjogren's syndrome or PTSD, for example. A trend can be detected by monitoring over a period of time. Linear discriminant analysis is but one type of multivariate statistical analysis. Other multivariate models can provide additional analytical insight into patters present in the data. For example, see FIG. 20.

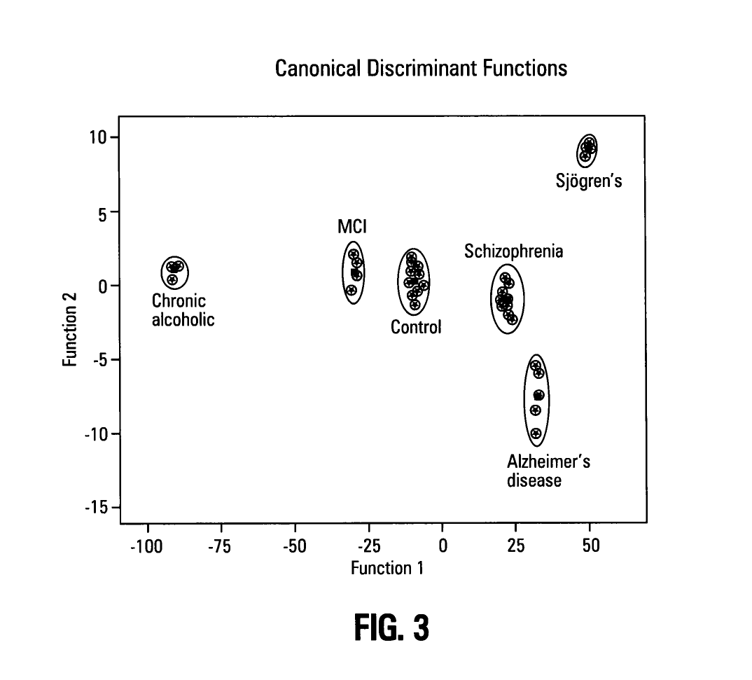

The plot shown in FIG. 3 illustrates results of the linear discriminant classification analysis of the zero-lag partial cross correlations according to one specific embodiment. FIG. 3 illustrates classification plots for 50 using 40 cross correlations selected using a genetic search algorithm. The group centroids are distinguished by tight clustering and clear separation. Beyond Linear discriminant analysis, other multivariate models can provide additional analytical insight into patterns present in the data. These methods include, but are not limited to Quadratic Discriminant Analysis, Principal Components Analysis, Cluster Analysis, Canonical Correlation, and Neural Network Classifiers, for example.

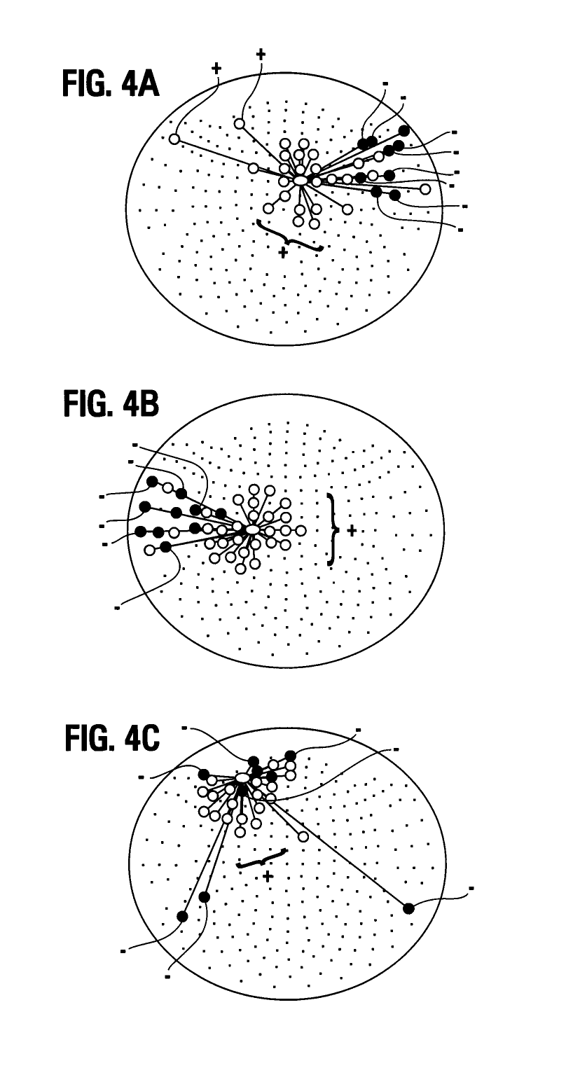

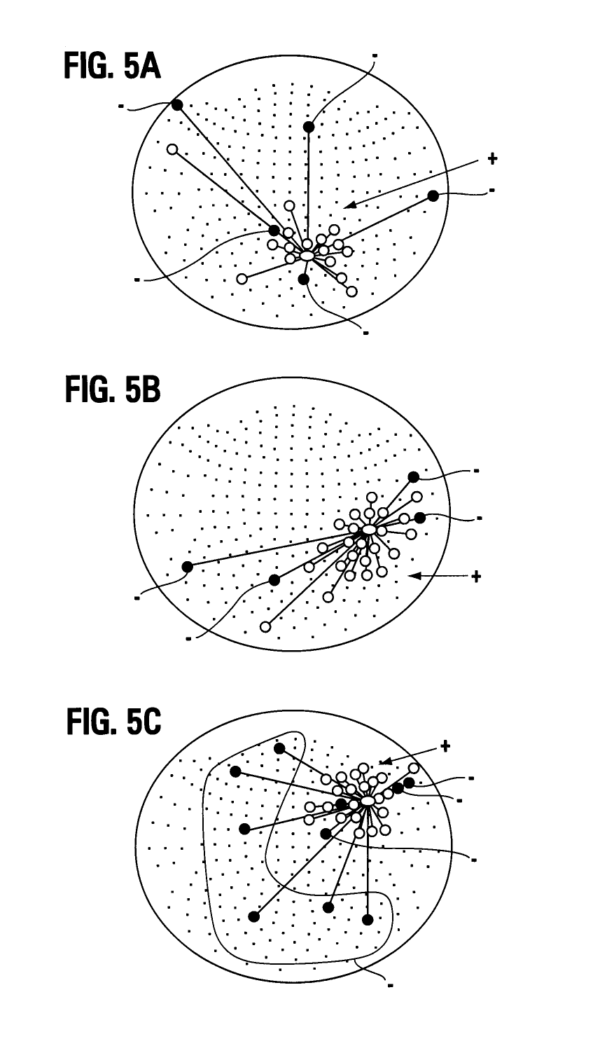

Exemplary empirical results are as follows: Given 248 sensors, a total of 248!/2!246!=30,628 PCC_{IJ}^{O} are possible per subject for a grand total of 30,628×10 subjects=306,280 PCC_{IJ}^{O}. Of those correlations, 285,502 (93.2%) were analyzed after excluding records with eye blink artifacts; 81,835/285,502 (28.7%) of those correlations were statistically significant (P<0.05). Of all valid PCC_{IJ}^{O}, 146,741 (51.4%) were positive and 138,761 (48.6%) were negative. The average (+SEM) positive Z_{IJ}^{O} was 0.0112.+−.0.00004 (maximum Z_{IJ}^{O}=0.38; PCC_{IJ}^{O}=0.36); the average negative Z_{IJ}^{O} was −0.0065.+−.0.00002 (minimum Z_{IJ}^{O}=PCC_{IJ}^{O}=−0.19). The absolute values of these means differed significantly (P<10⁻²⁰; Student's t test), the average |+Z_{IJ}^{O}| being 72% higher than the average |−Z_{IJ}^{O}|. Examples of spatial patterns in the distribution of synchronous coupling between a sensor and all other sensors are illustrated in FIGS. 4A-4C and 5A-5C. Only statistically significant PCC_{IJ}^{O} are plotted. The statistical significance threshold was adjusted to account for 247 multiple comparisons per plot, according to the Bonferroni inequality: the nominal significance threshold is P<0.05, corresponding to an actual threshold used of P<0.05/247 (i.e., P<0.0002). Positive and negative PCC_{IJ}^{O} are indicated. Small dots represent the location of the 248 sensors, projected on a plane. Data are from one subject.

Relation between PCC_{IJ}^{O} and intersensor Distance. Overall, PCC_{IJ}^{O} varied with the distance, dᵢⱼ, between sensors i and j. In general, sensors closer to each other tended to have positive PCC_{IJ}^{O}. The average intersensor distance dᵢⱼ for negative zᵢⱼ^{o} was 24% longer than for positive zᵢⱼ^{o}. Specifically, dᵢⱼ (−zᵢⱼ^{o} was 198.92.+−.0.21 mm (n=138,700), and dᵢⱼ^{o} (−zᵢⱼ^{o} was 160.12.+−.0.24 mm (n=146,675). Overall, there was a strong and highly significant negative association between zᵢⱼ^{o} and the log-transformed dᵢⱼ, ln(dᵢⱼ). The Pearson correlation coefficient between signed zᵢⱼ^{o}, and ln(dᵢⱼ) was −0.519 (P<10⁻²⁰). This relation indicates that the strength of synchronous coupling tended to fall off sharply with intersensor distance.

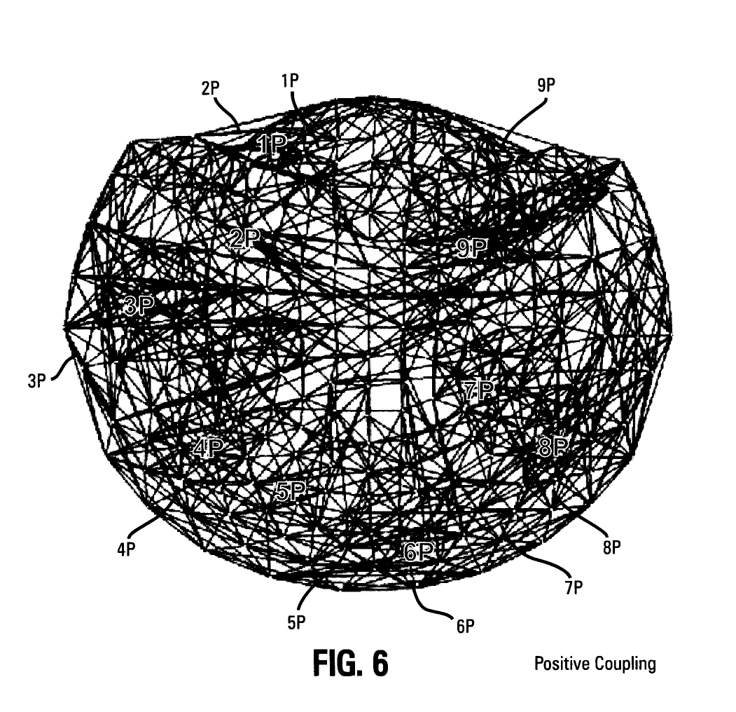

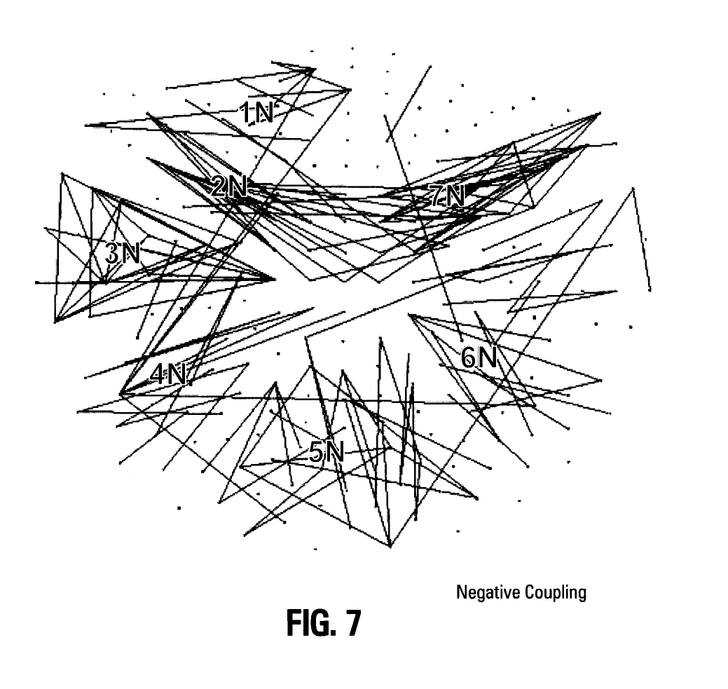

Synchronous Dynamic Neural Networks. The PCC_{IJ}^{O} is an estimate of synchronous coupling between neuronal populations in which the absolute value and PCC_{IJ}^{O} denote the strength and kind of coupling, respectively. If the neural ensembles sampled by the 248 sensors are considered nodes in a massively interconnected neural network, such as the massively connected neural network map visualized in FIGS. 6-7, then the PCC_{IJ}^{O} can serve as an estimate of the dynamic synchronous interactions between these nodes. Such a massively interconnected network can be visualized by connecting the 248 nodes with lines and indicating whether each line represents a positive or a negative coupling. FIGS. 6 and 7 show a thresholded and scaled view of this network, averaged across the 10 subjects; regional variations in interactions were present and consistent across subjects.

Features noted in this network include: (i) most of the next-neighbor interactions are positive; (ii) most of negative interactions occur at longer distances; (iii) interactions with centrally located sensors are relatively sparse; and (iv) inter-hemispheric interactions are infrequent, probably because of the longer distances involved. In addition, systematic variations in the local density of interactions can be distinguished qualitatively, as follows (in counterclockwise direction). There were nine regions of positive interactions (FIG. 6), consisting of sensors overlying the following brain regions: left anterior-frontal (1P), left dorsal-frontal (2P), left lateral-frontal-temporal (3P), left parietal (4P), left parietal-occipital (5P), right occipital (6P), right parietal-temporal (7P), right temporal (8P), and right frontal (9P). For negative interactions (FIG. 7), seven regions could be distinguished, consisting of sensors overlying the following brain regions: left anterior-frontal cortex (IN), left dorsal-frontal (2N), left lateral-frontal-temporal (3N), left parietal (4N), occipital (5N), right parietal (6N), and right frontal (7N). Several of the positive and negative interactions were spatially overlapping.

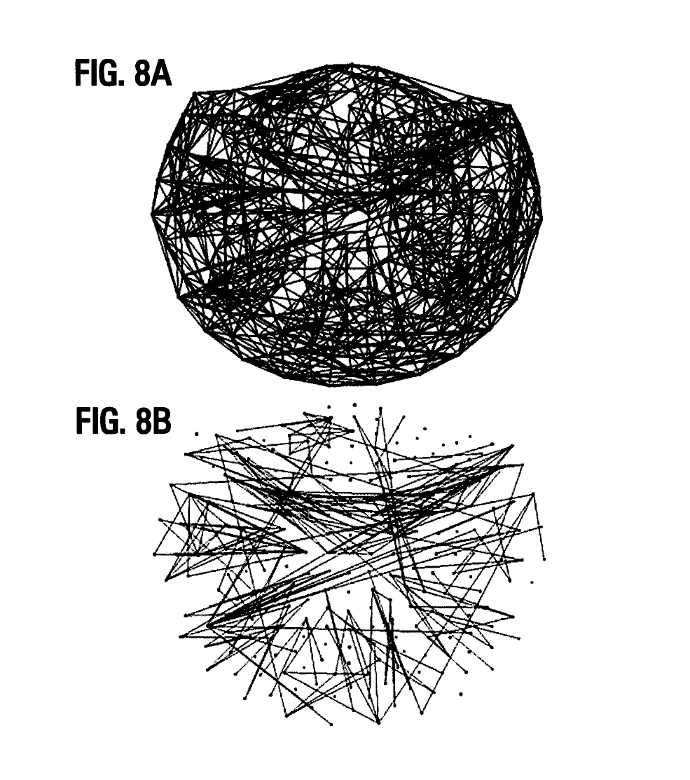

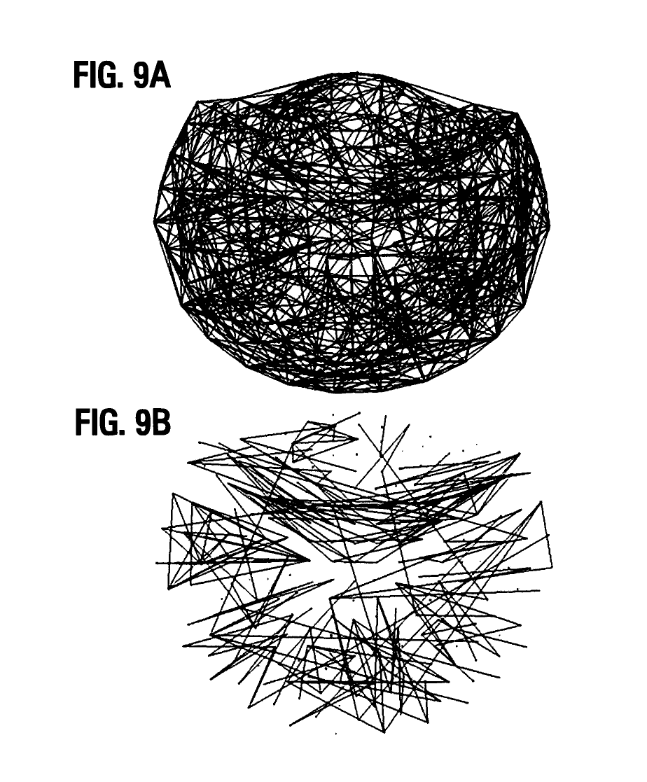

Notably, neural networks constructed as above were very similar across subjects (FIGS. 8A and 8B, and 9A and 9B). Overall network similarity can be quantified and assessed between all subject pairs by calculating the Pearson correlation coefficient across all zᵢⱼ^{o} (i.e., all i and j sensors) of the network, for example. The correlation coefficients obtained are high and highly significant (median=0.742; range, 0.663-0.839; P<10-20 for all correlations; >20,000 degrees of freedom). These findings suggest a common network foundation.

In previous studies, associations between neuronal ensembles (recorded as EEG, MEG, or local field potentials) have been investigated by using frequency-domain or time-domain analyses applied to a whole data set or within specific spectral frequency bands. In such analyses, association measures are commonly calculated from the data without testing for their stationarity. Stationarity (or quasistationarity) provides accurate measurements of moment-to-moment interactions between time series (as contrasted to shared trends and/or cycles), both in the time domain (by computing cross correlation) and in the frequency domain (by computing squared coherency). In contrast, cross correlation or coherency estimates based on raw nonstationary data may yield erroneous estimates and spurious associations.

The sign of cross correlation does not provide information regarding underlying excitatory or inhibitory synaptic mechanisms but merely indicates the kind of simultaneous covariation with respect to the mean of the series: a positive correlation indicates covariation in the same direction (increase/increase, decrease/decrease), whereas a negative correlation indicates covariation in opposite directions (increase/decrease, decrease/increase). In general, PCC_{IJ}^{O} tend to vary in an orderly fashion in sensor space, such that it tends to be positive between neighboring sensors and negative between sensors farther away. Although this tendency was noted, there are clear and distinct exceptions, including negative PCC_{IJ}^{O} between neighboring sensors and positive PCC_{IJ}^{O} between far-away sensors. In addition, the spatial PCC_{IJ}^{O} pattern differed depending on the location of the reference sensor. The findings suggest a robust and relationally orderly correlation structure, but with distinct local specificity. Indeed, these characteristics are the fundamental attributes that endow the resulting massively interconnected network with the characteristic structure illustrated in FIGS. 6-9B.

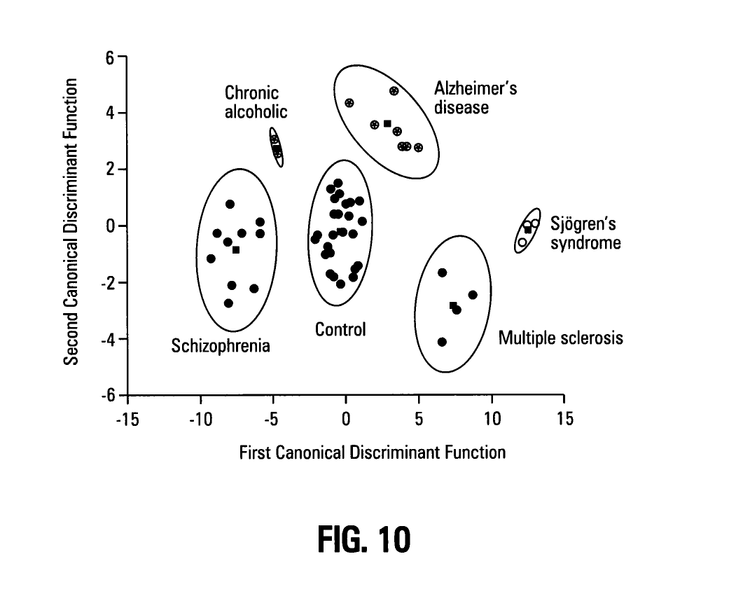

A statistically significant Group effect on zuᵢⱼ^{o} (P<0.05, F test, ANCOVA) was found in 18% of sensor pairs. Next, a linear discriminant classification analysis (using GA) was carried out on subsets of zᵢⱼ^{o} to find out whether individual subjects can be successfully classified to their respective groups. Indeed, many (in the thousands) such subsets of zᵢⱼ^{o} predictors that classified each one of the 52 subjects 100% correctly were found. (The exact number of all such subsets is practically impossible to be determined.) An example is shown in FIG. 10. In many cases, successful subsets yielded not only 100% correct classification but also high (e.g., >0.98) posterior probabilities of correct classification of each subject to its group.

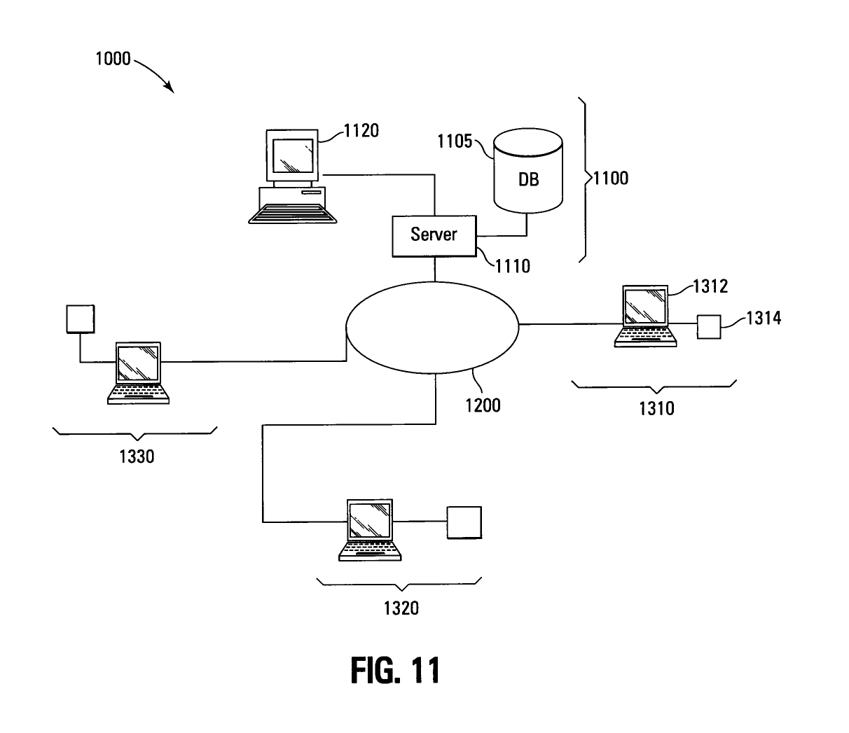

In one example, the present subject matter includes a system and method for processing obtained sensor data to produce dynamic models. FIG. 11 illustrates system 1000 including central server 1100 and communication network 1200. Central server 1100 includes server 1110 coupled to database 1105 and terminal 1120. Server 1110 executes and algorithm based on instructions stored in a memory or other storage facility such as database 1105. Database 1105 can include magnetic, optical, flash, or other suitable data storage device. Terminal 1120 provides an input device as well as an output device to allow operation and control of system 1000.

In FIG. 11, client sites 1310, 1320 and 1330 are representative of clinics or health care facilities that generate data according to the present subject matter. Three such client sites are illustrated however, more or fewer are also contemplated. Data, for example, is generated at client site 1310 by sensor 1314 under control of local processor 1312. The data includes a time series corresponding to brain activity. The time series is captured using local processor 1312 in communication with sensor 1314 which can include an array of superconducting quantum interference devices (SQUIDS). Time series data stored at local processor 1312 is communicated to central server 1100 using communication network 1200. Communication network 1200 can include a wired or wireless network, examples of which include an Ethernet network, a local area network (LAN), a wide area network (WAN) such as the Internet, and a public switched telephone network (PSTN).

The central server can include a processor coupled to a memory and having instructions stored thereon to execute an algorithm as described herein. The central server can include more than one processor which can be distributed across multiple locations. Persons skilled in the art will readily appreciate that the processor of the central server can be embodied by any suitable processor including, without limitation, a RISC or CISC microprocessor, a microcontroller, a microcomputer, a FPGA, an ASIC, an analog processor circuit, a quantum computer, or a biological processor, for example, and can include single or multiple processing units. The processor can also be of a type that operates in batch mode or real-time mode.

In one example, client sites are licensed or enrolled on a subscription basis. For a fee, the central server executes an algorithm to generate an estimate of dynamic brain activity based on the time series. In one example, the central server provides a report which includes the estimate. The estimate can be rendered in an alphanumerical or graphical format.

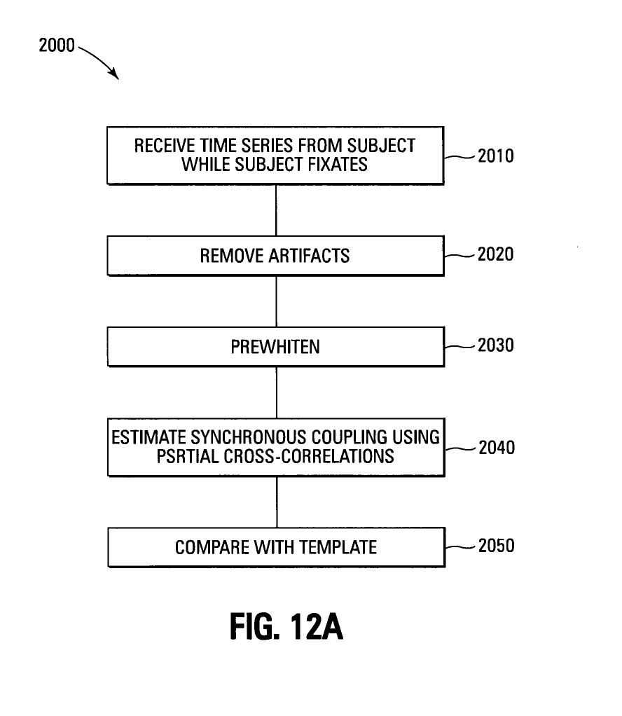

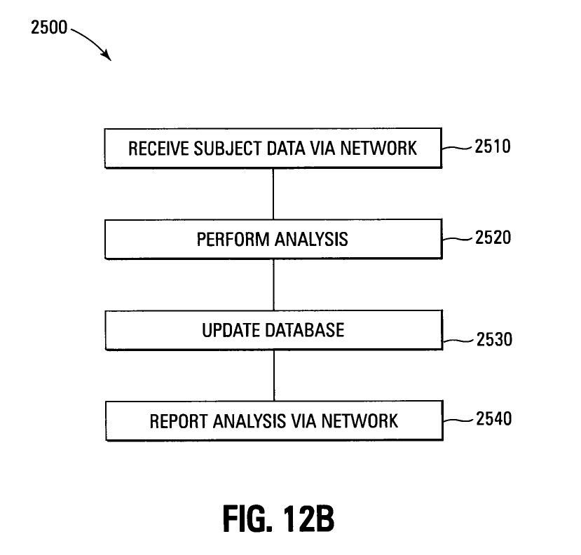

FIG. 12A illustrates method 2000 performed by one example of the present subject matter. At 2010, time series data is received. The time series data is generated while the subject is performing an eyes-open task involving only nominal stimulation and motor activity, such as visually fixating on a target. This type of eyes-open task causes the subject's brain to remain in a generally idle state. Note that eyes-closed tasks, or non-idle tasks are contemplated as well, and may be utilized in generating data for some applications.

The time series data can be received and stored by a processor some time after the data is generated by a sensor or array of sensors. At 2020, artifacts in the data are removed. Artifacts can include those produced by breathing, cardiac artifacts, physical movement or other artifacts. At 2030, the data is prewhitened by, for example, converting the MEG time series to a stationary, white noise series. At 2040, an estimate of synchronous coupling is generated by calculating partial cross correlations. The estimate is then compared with a template at 2050.

In one example, the template is generated based on stored data for the particular subject under review. In one example, the template is generated based on stored data derived from a plurality of different subjects. An analysis can be performed by comparing the subject data with a template, and, in one example, the template is modified with the results for that particular subject. In another example, the template is modified in a batch mode after having compiled a number of subjects over a period of time.

FIG. 12B illustrates method 2500 suitable for implementation using a network such as that shown in FIG. 11. At 2510, the subject data is received over an internet connection. In one example, the subject data includes the MEG time series data. At 2520, analysis is performed using, for example, server 1100. At 2530, database 1105 is updated with the information corresponding to the particular subject. At 2540, the results, which can include analysis of the data, are reported to the client site using the network.

In one example, the central server provides a screening report that provides an indication of normalcy. Such a binary report, showing normal or a departure from normal, can be used as a threshold determination by the client site as to brain condition.

In one example, the central server can provide a diagnosis that includes a classification based on a comparison with a database. The database includes stored data corresponding to a number of previously analyzed time series. In addition, the database can be updated with new data as client time series data is received. In one example, the client site can request and receive trend data that includes a comparison of earlier time series data for a particular brain with later time series data. In forming a diagnosis, the present subject matter differentiates among a plurality of disease states.

The database can provide data for generating a template or model for analysis of a particular subject. A template can, for example, correspond with a particular disease or other neuronal condition or with a normal brain.

In one example, the central server provides feedback to allow monitoring of subject progress. In particular, disease progression and therapy progression can be monitored by generating multiple estimates over a period of time. In addition, estimates of neuronal synchronicity can be generated during a drug trial. Safety and efficacy of a therapy regimen can be evaluated by using the present subject matter to monitor a drug trial.

A computer implemented algorithm can be implemented in software instructions stored in a memory. Portions of the software can be executed at a client site and the central server.

In one example, the estimate is determined, in part, as a function of the age of the subject. Age-adjusted data can be stored in the database. Other data can also be stored in the database and used for discriminating, including, for example, known medical conditions or therapy regimens.

As the database evolves, it is expected that particular variables will be strongly correlated with particular disease conditions. As such, these particular variables can be weighted differently to more quickly and accurately distinguish between different conditions. In one example, subjects can be classified using a subset of the calculated correlations as a predictor. For example, a linear discriminant classification analysis using the ‘leave-one-out’ method can be used. In one example, six correlations are adequate to correctly classify subjects (100% correct) with posterior probability of 1.0. Linear discriminant analysis is but one type of multivariate statistical analysis. Other multivariate models can provide additional analytical insight into patters present in the data.

In addition, it is believed that embodiments the present invention may have utility for discerning the veracity of a subject. In the form of a lie detector, data is collected from the subject coincident with an assertion to be tested. In addition, it is believed that the other embodiments of the present invention may have utility for analyzing or testing intelligence. As such, particular markers may be identified to coincide with a particular intelligence grade.

Certain embodiments of the present invention can provide an objective test to enhance diagnostic accuracy, advance the recognition of AD (and other conditions) into a presymptomatic stage, and serve as a monitor for therapy.

The number of sensors used to capture the time series can be adjusted to any value and in one example the number is reduced to a value sufficient to reach a conclusion of interest. For instance, one example uses a reduced set of sensors, (i.e. six or fewer) to generate a meaningfully time series sufficient to reach a conclusion as to a particular neurological condition.

In general, embodiments of the present invention can be used to diagnose a condition or disease using a stored template, differentiate between a number of different conditions, different effects of drugs, or different diseases, and monitor a subject over a period of time.

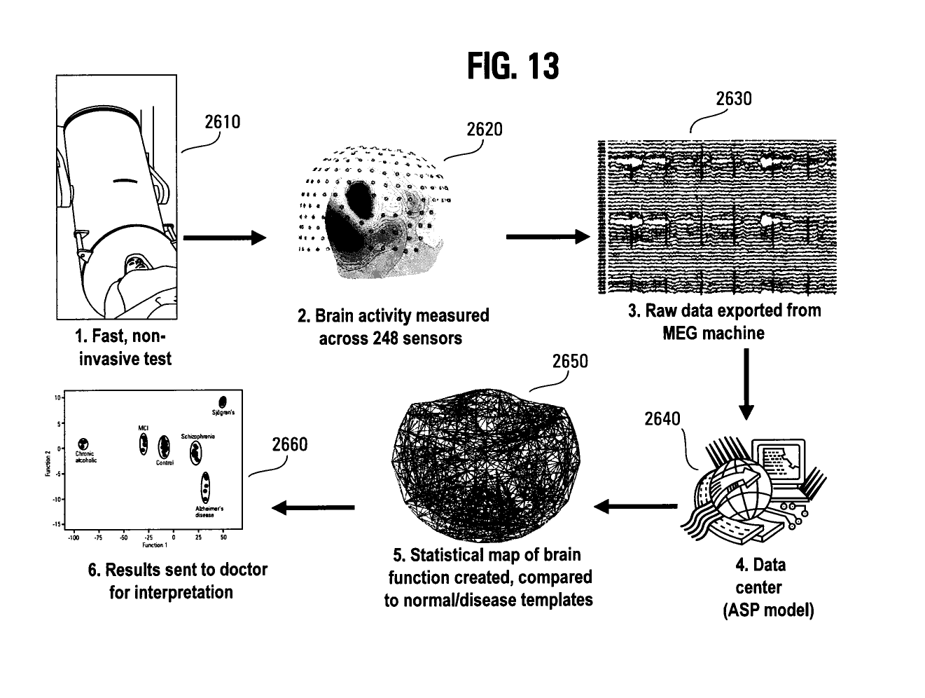

FIG. 13 illustrates an overview of a process according to one embodiment. At 2610, an electromagnetic measurement apparatus, such as a MEG conducts a non-invasive test of a subject. As described above, in one embodiment, the subject is instructed to perform an eyes-open fixed visual stimulus task to place the subject's brain in an eyes-open idle state. At 2620, the electromagnetic measurement apparatus gathers time series data of the patient's brain. In one embodiment, the data is sampled at a minimum sampling frequency of 1 kHz corresponding to a time resolution of 2 ms or better. This relatively fast sampling rate and temporal resolution generally corresponds to the rate at which neural activity occurs in the subject's brain. The data is gathered by a multiplicity of sensors spatially distributed around the subject's brain.

At 2630, a set of time series, each of which has been gathered by a corresponding sensor is transmitted or otherwise delivered to a data center which has data processing and, optionally, data storage facilities. At 2640, the data is received at the data center. The processing occurring at 2650 produces a dynamic model that represents statistically-independent temporal measures among neural populations of the subject. The temporal measures can be, for example, time-wise related sensed signals detected by various sensors. These signals may coincide based on the sampling intervals such that they have coincidence without lag (i.e., simultaneous, or asynchronous by less than a detectable amount). Alternatively, the temporal measures can be based on non-synchronous, but nevertheless temporally-related signals, such as signals interacting within a certain time window (e.g., a 50 ms window).

The statistical independence of the temporal measures relates to the apparent interaction between the interaction groupings (e.g., pairings) of sensors taking into account the other variables. One type of computation that can achieve statistically independent temporal measures is the partial cross correlations described in the above examples. However, other approaches may be applicable in certain applications within the scope and spirit of the invention. For instance, the use of residuals may produce statistical independence of interaction groupings of temporal measures.

As discussed above, the dynamic nature of the model means that the model of temporal measures is represented as a function of time, such that it can be different for each sampling period. Notably, the dynamic model of temporal measures can be regarded in one sense as a network of interacting spatial nodes, and not merely a network having nodes in only a structural configuration. While the above examples provide spatial representations of the “brain maps,” the data can be represented in any suitable form within the scope and spirit of the invention.

As described above, certain advantages may be realized in processing the raw measurement data to remove artifacts and/or to pre-whiten each of the time series to produce signals having a characteristic of stationarity of mean, variance, and autocorrelation. This step of pre-whitening further contributes to the statistical independence of the temporal measures that are to be computed.

Once the dynamic model is computed, it can be further processed to simplify or filter the model. One type of filtering is the use of a threshold function to remove temporal measures having a relatively weaker magnitude, and leaving only the strong temporal measures to utilize for analyzing the subject's brain.

In one embodiment, temporal measures are analyzed for covariance with one or more external property of the subject such as, for example, age, race, or neuropsychological capacities.

At 2650, the data center compares the dynamic model of temporal measures with one or more templates classified according to various brain conditions. Templates can be regarded in one sense are validated models of neurophysiologic conditions. In one type of embodiment, templates are each based on a group of previously-evaluated subjects that share a common neurophysiologic characteristic, such as a disease, disability or, more generally, condition. In this embodiment, the templates are validated in that there is strong statistical correlation among indicators corresponding to the condition for the group of subject upon which the template is based.

Each template may itself be a dynamic model of temporal measures, or a subset of such a dynamic model. A template may be stored as a data record, or may be represented as an algorithm or function that, when “compared” to the subject's dynamic model, modifies the dynamic model to achieve the result of the comparison. In one sense, a template is a classification function. In one example embodiment, a template is in the form of a data mask with weighted taps.

As in the examples above, the template can be limited to only a selected subset of interaction groupings (e.g., pairs) of temporal measures, with the remaining temporal measures omitted as being irrelevant to the condition to which that template corresponds. In this regime, different templates may have different interaction groupings of relevant temporal measures to the corresponding condition or disorder.

When the dynamic model (or subsets thereof) of patient data is compared against one or more templates different subsets of the dynamic model may be compared against each different template. Thus, for instance, in a template that represents pairs A, B and E of correlated sensor data (identified based on their spatial positioning), only pairs A, B, and E of the dynamic model of temporal measures taken from the subject need to be compared. For a different template in which pairs C, D, and E are relevant, only those pairs taken from the dynamic model may be used. The resulting comparison can be scored, or otherwise represent a degree of correlation. Alternatively, the comparison can produce a binary (yes/no) result.

In one aspect of the invention, the dynamic model of the temporal measures of the subject is stored, and later used to compare against more recent measurements of the same subject. This approach may be useful for tracking disease progression or evaluating effectiveness of a particular therapy. In a related embodiment, a template is made based on different sets of data from the same subject, and the template is used for tracking of the patient's condition over time.

At 2660, the system generates a report, which may include a graphical representation of the dynamic model of the patient, mapped to 2-d or 3-d space for visualization similar to the output illustrated in FIG. 3 or 10.

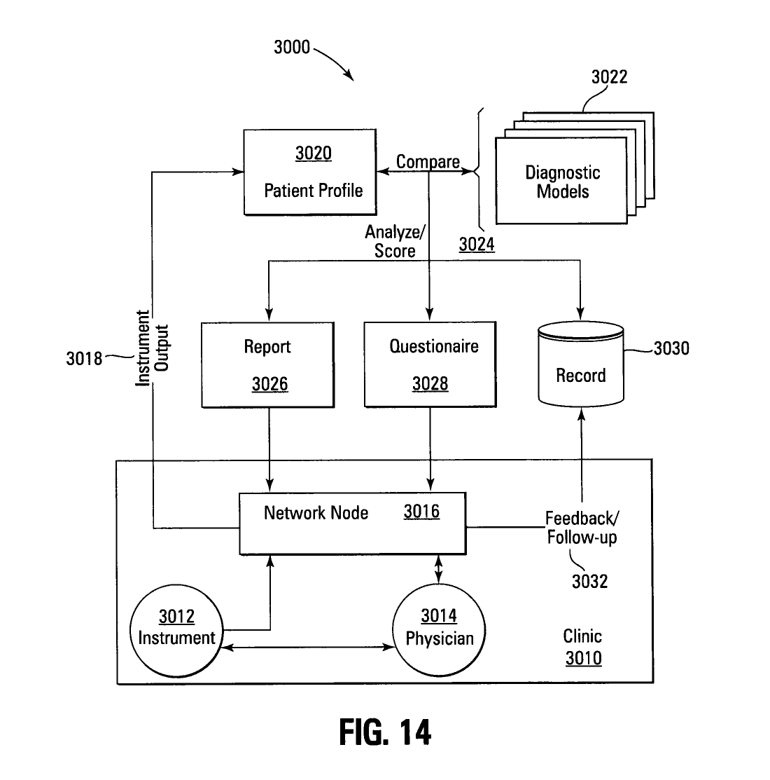

FIG. 14 is a diagram illustrating information flow 3000 according to one aspect of the invention. Clinic 3010 includes a subject-measuring instrument 3012, and physician or lab technician 3014. Network node 3016 facilitates communication with remote nodes. In one embodiment, the network node 3016 includes a computer system, such as a PC, having a network interface. Network node 3016 can also facilitate an operator interface between physician 3014 and the instrument 3012.

In one embodiment, measurements are made by instrument 3012 and stored locally on network node 3016 prior to transmission. Network node is then instructed to transmit instrument output 3018 to an external system for analysis. The system creates a patient profile 3020 corresponding to instrument output 3018 in association with a patient ID. The system processes information from patient profile 3020, such as the instrument output 3018, according to any of the analysis techniques described above, and including comparing information based on the instrument output against diagnostic models 3022. In one embodiment, diagnostic models 3022 are analogous to the templates described above.

The result 3024 of the comparison can be used to generate report 3026 for delivery to clinic 3010 via network node 3016. Report 3026 can include the result 3024 of the comparison, along with an automatically-generated discussion and graphical output depicting the result 3024. Additionally, the result 3024 can be associated with questionnaire 3028, also for delivery to clinic 3010 via network node 3016. Questionnaire 3028 can be filled out by physician 3014 to provide additional information of interest about the patient, the testing environment, therapies, manual diagnoses, and the like. The filled-out questionnaire is then provided as feedback/follow-up 3032 to be stored in data store 3030 in association with the patient ID, the report 3026, the result 3024, and the instrument output 3018.

### Regional Analysis

Embodiments of the present invention utilize a regional analysis approach for evaluating brief, resting state brain scans, such as MEG, EEG, fMRI, or fPET scans, for example. This approach is founded the SNI data processing and analysis according to the embodiments described above, and variants thereof.

In one embodiment, raw brain scan data, such as the data produced by MEG or EEG measurement, is processed and analyzed as described above, which in certain embodiments produces zero-lag correlation values for all interaction sets (e.g., pairs) of sensors in the sensor array.

In one type of embodiment, regional analysis is based on spatial divisions. Accordingly, the MEG or EEG sensors are assigned into groups located over various human brain spatial regions. These brain regions may be contiguous regions that are similar to eight regions described typically in the scientific literature, or may be any other defined set of brain regions.

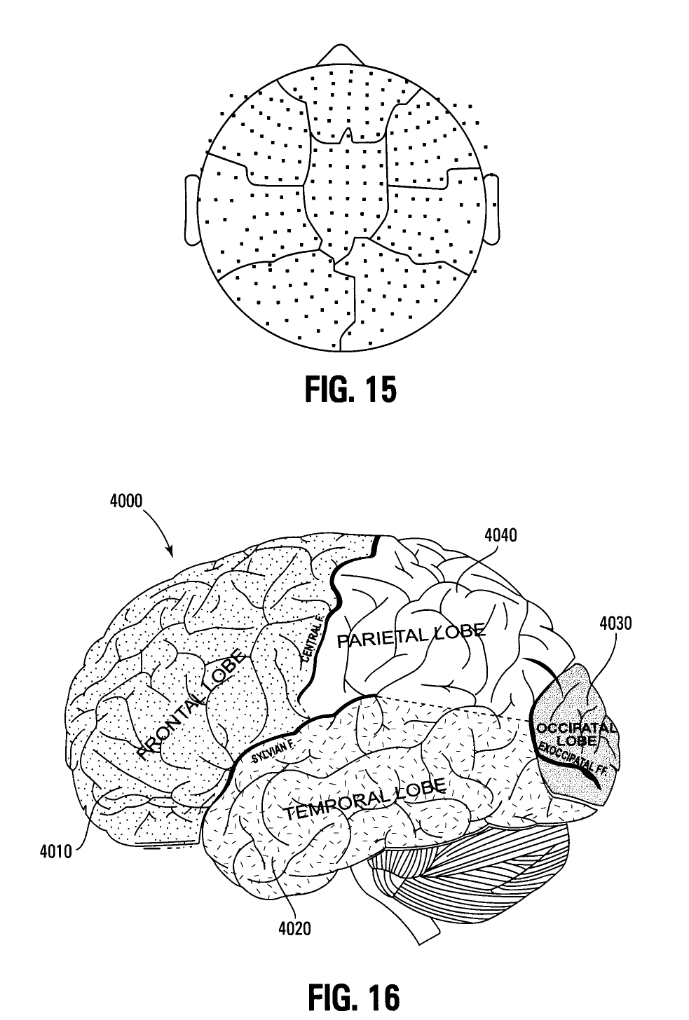

FIG. 15 illustrates an example set of brain regions as defined according to one embodiment, and depicts regional groupings of MEG sensors situated over those regions. Shown in this example embodiment is a map of sensor locations (small dots) corresponding to the sensor array in a 4D Neuroimaging WH3600 Gradiometer MEG instrument in relation to a human head. Each area delineated by the lines represents a corresponding one of eight regions.

In the example shown, for the 4D Neuroimaging WH3600 MEG instrument, eight regions are defined; however, the number of regions can be defined differently. For example, in another embodiment, the regions are defined based on brain structure.

Referring to FIG. 16, an example set 4000 of structure-based brain regions is depicted. In the embodiment shown, a group of sensors encompasses those only near the frontal lobe 4010. Another group encompasses those sensors which are only near the temporal lobe 4020. Yet another group encompasses sensors only near the occipital lobe 4030. Yet another group encompasses sensors only near the parietal lobe 4040. In this manner, regions based on brain structures can be defined.

In a related embodiment, each region corresponds to a functional center of the brain. In one example, the different regions may include the speech center, the motor control center, the hearing center, the smell sensing center, the touch and pressure sensing center, the taste sensing center, the vision center, the language center, and the like.

FIGS. 20A-20F depict representative source code for defining regions and calculating regional values, according to one embodiment. A series of values are calculated from the original set of pair-wise correlations that are obtained using the techniques and apparatus described in existing temporal approach as described above. In one embodiment, the aggregated values are spatially aggregated such that one value represents synchronous neural activity for a corresponding region, or for an interaction between a specific set of regions. In another type of embodiment, the aggregated values are temporally aggregated, such that a single value represents activity occurring over a period of time. In a related embodiment, the aggregated values are both, spatially, and temporally aggregated.

In one example embodiment, a total of 41 aggregated values are calculated. These aggregated values can be used, for example, to define specific alterations in brain function associated with disease or the effect of neuroactive drugs.

### EXAMPLES

The following exemplary process demonstrates a regional analysis according to one embodiment. In this example, the terms “mean”, “median”, “mode,” “average” “proportion,” “variance,” “standard deviation,” and the like, have their standard mathematical definitions.
1) A dynamic brain model is generated according to the techniques described above for all groups (e.g., pairs) in the sensor array.
2) Regional groupings of contiguous sensors are established. For example, referring to FIG. 15, the regional groupings are defined roughly according to major brain regions. Alternative definitions for groupings are contemplated in various related embodiments, such as by functional area, brain structure, distance, etc. The regional groupings in various embodiments may or may not be contiguous as in this example.
3) Aggregated correlation values for all interaction sets (e.g., pairs of sensors) within each individual region are calculated to generate N separate region representative values, where N is the number of regions. For the interaction sets, the correlation values (or the squared correlation, or the Fisher transformed correlation values), can be aggregated using any of a number of suitable techniques, such as taking a mean of the values for the region, a median value, a mode value, sum of the absolute values, or the like. Additional selection may be performed (such as removing outlier values—e.g., those beyond X standard deviations from the mean value for the group, then recomputing the mean value on the resulting set).
4) Aggregated cross-regional correlation values are calculated for each interaction set (e.g., pair) representing the different regions of interest. By way of example, consider a pair of regions to be analyzed for cross-regional correlation, Region A, and Region B. Assuming each region has M sensors (sensors A₁ . . . A_{M} and B₁ . . . B_{M})—although it should be noted that there do not have to be an equal number of sensors in each of the regions—the cross-region interactions between pairs of sensors in this embodiment would be the interactions (A₁-B₁, A₁-B₂, . . . , A₁-B_{M}, A₂-B₁, . . . , A_{M}-B_{M}). These interactions are then aggregated as in (3) above.
5) Two global measures are generated for a) sets of sensor interaction sets in which the distance between the sensors of the interaction group are closer than the median distance (Short); and b) groups of sensor pairs where the sensors of the interaction group are equal to or farther apart than the median distance (Long). In variations of this approach, a larger number of distance groupings may be used.
6) Three additional global measures are calculated based on a) the proportion of sensor interaction sets having correlation values significantly less than zero (e.g. less than −0.01), the proportion of sensor interaction sets having correlation values significantly greater than zero (e.g. greater than 0.01) and 3) the proportion of sensor interaction sets that are not significantly different from zero (between −0.01 and 0.01).

In one specific example using 4D Neuroimaging WH3600 gradiometer instruments having 248 sensors, and using pairs of sensors as the interaction sets, 30628 unique sensor pairs are analyzed. The sensor array is divided into N regions (e.g., N=8 regions each having 31 sensors). There are 465 (31×30/2) pairs within each region and 961 (31×31) individual sensor pairs between any two regions. For a regional analysis involving 8 regions there are 28 cross-region values and 8 within-region values in total.

According to various other embodiments, the following adaptations of the above-described techniques can be implemented:
1) Use non-zero lag correlation values as input data for the regional analysis;
2) Use cross-covariance values instead of cross-correlation values;
3) Use different numbers of regions with equal or unequal numbers of sensors per region;
4) Calculate and use any suitable form of statistical aggregation for within-region and cross-region attributes such as, for instance, the a sum—e.g., a sum of absolute values, a median or the median squared correlation value, the use of normalized values, etc.;
5) Calculate a proportion of negative, zero and positive correlations for each set of within-region or cross-regions sensor pairs Clinical studies were conducted to collect resting-state MEG scans and associated clinical evaluations for volunteer subjects older than 55 who were healthy or had previous diagnoses of Alzheimer' disease (AD) or mild cognitive impairment (MCI). MEG data from one minute, eyes open resting-state scans was processed and analyzed separately in 8 predefined groups of sensors (31 sensors each group) using a regional variant of Synchronous Neural Interaction (SNI) Test according to aspects of the invention. Eyes-closed states are contemplated as well, but not in this example. A subset of subjects were evaluated and scanned two times separated by approximately 9 months to assess changes associated with disease progression.

Region-specific, functional changes associated with diagnosis were observed and normalized results from some regions are summarized in Table 1.

TABLE 1
Summary of SNI regional analysis

| Group | N | RPar | C-RTemp | LPar-RPar | Short |
|---|---|---|---|---|---|
| Healthy | 71 | 0.34 ± 0.022 | 0.34 ± 0.023 | 0.39 ± 0.019 | 0.33 ± 0.022 |
| MCI | 24 | 0.40 ± 0.033 | 0.29 ± 0.034 | 0.48 ± 0.030 | 0.40 ± 0.034 |
| AD | 82 | 0.46 ± 0.022 | 0.42 ± 0.022 | 0.53 ± 0.021 | 0.44 ± 0.021 |

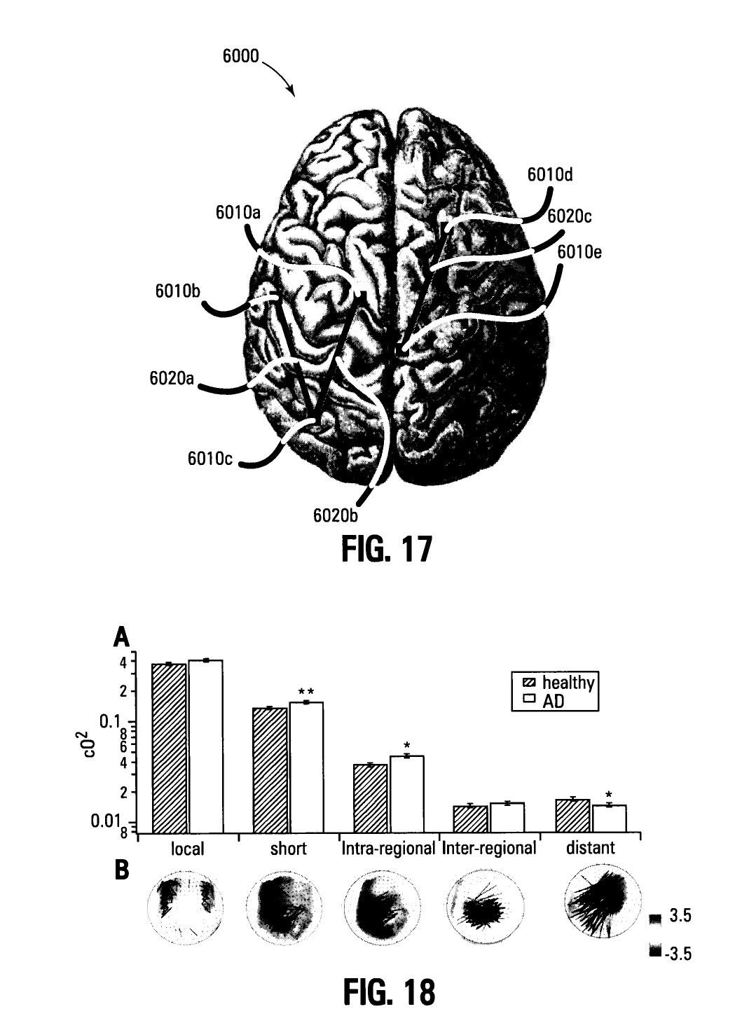

Referring to FIG. 17, an embodiment 6000 of a distance-based regional grouping of sensor pairs is depicted. In this illustration, sensor 6010a is related to sensor 6010c to form distance 6020b. Similarly, sensor 6010b is related to sensor 6010c to form distance 6020a. Also, sensor 6010d is related to sensor 6010e to form distance 6020c. In this embodiment, distance 6010a, distance 6020b, and distance 6020c are all equivalent. The regions are summarized in the following Table 2.

TABLE 2
Distance-Based Regional Grouping of Sensor Pairs

| Sensor 1 | Sensor 2 | Distance |
|---|---|---|
| 6010a | 6010c | 6020b |
| 6010b | 6010c | 6020a |
| 6010d | 6010e | 6020c |

In one example utilizing distance-based regional groupings, detailed analysis of the observed changes in functional connectivity revealed that AD was associated with widespread alterations in the strength of communication as represented by c0² values in Table 3. It was particularly informative to examine how AD altered connectivity between pairs of sensors separated by different distances. Referring to FIG. 18, AD patients show increased functional connectivity between nearby sensors and decreased connectivity between distant sensors. The bars in subpart A represent the mean±SEM c0² value (representing strength of correlated activity) for each of five sets of sensor pairs defined by the distance between them. Heat maps in subpart B represent the T-values calculated from standard t-tests conducted on each sensor pair. For each map only those pairs within the corresponding distance group are mapped. (* −p<0.01; **−p<0.001). AD increased the strength of correlation between short and intra-regional sensor pairs over large parts of the sensor array. By contrast, the strength of correlation between distant sensors was significantly lower in the AD group, especially between pairs of sensors in the left parietal and right frontal regions.

TABLE 3
Relationship to neuropsychological testing - cross sectional

| | ADAS-cog | | CDR | | MMSE | |
|---|---|---|---|---|---|---|
| | Prop¹ | R range² | Prop | R range | Prop | R range |
| Delta | 0.12 | −0.11-0.30 | 0.01 | −0.1-0.17 | 0.03 | −0.22-0.19 |
| Theta | 0.95 | 0.10-0.41 | 0.96 | 0.11-0.42 | 0.93 | −0.41-−0.12 |
| Alpha | 0.42 | −0.39-0.09 | 0.16 | −0.24-0.07 | 0.19 | −0.16-0.35 |
| Beta | 0.69 | −0.43-0.01 | 0.24 | −0.25-0.02 | 0.17 | −0.07-0.31 |
| Gamma | 0.31 | −0.31-0.09 | 0.09 | −0.21-0.1 | 0.03 | −0.19-0.25 |
| c0² | 0.07 | −0.37-0.33 | 0.08 | −0.36-0.30 | 0.02 | −0.32-0.33 |

The proportion of MEG scan features that showed a statistically significant cross-sectional correlation between the indicated neuropsychological test and MEG scan feature. The range of Pearson correlation coefficient values found for the indicated MEG scan feature and neuropsychological test score

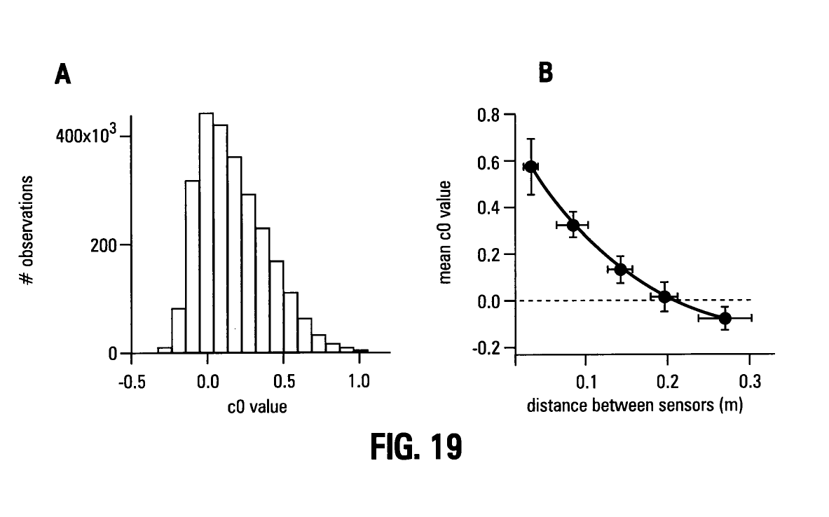

Referring to FIG. 19, the magnitude and direction of c0 values varies regularly with the distance between sensors. Shown in subpart A is the distribution of all c0 values from all sensor pairs and all healthy control subjects (bin width=0.1). Each subject scan generated 20910 c0 values (n=123 subjects in the healthy control group). In subpart B, the network of c0 values was divided into five groups based on the distance between the sensor pairs. Individual c0 values for within each distance group were averaged for each subject. The markers represent the average of these distance-group c0 values plotted as a function of average distance between sensors. Y error bars represent the standard deviation across the healthy control group for each of the five distance groups and the X error bars represent the standard deviation of the distances within each group.

There are a number of advantages of the regional approach including, for instance, reduction of data dimensionality, decreased variability between subjects and instruments, and an ability to implement empirical testing of predefined hypotheses regarding regional changes in brain function.

The reduction of data dimensionality can offer significant reduction of feature dimension—e.g., summarizes the 30,000+ features into 36 regional features (8 intra-region and 28 inter-region correlations. The dimension reduction facilitates multivariate model building for disease signature and treatment effect by potentially protecting against overfitting.

The decrease in variability reduces various confounds, such as those due to head movements or physical positioning of the subject's head relative to the measuring instrument's sensors, since the regional analysis summarizes activity over a broader cortical region than that of a single sensor which would otherwise be significantly affected by movement. Separately, the regional approach allows comparisons across different measurement systems with different numbers of sensors and sensor montages.

Notably, the regional approach enables human researchers to perform empirical testing of predefined hypotheses regarding regional changes in brain function. Thus, for instance, researchers may hypothesize that an effect of a certain psychoactive drug will impact long-distance interactions between neural populations in the brain, and the dynamic modeling using a regional analysis can specifically test for such effects.

Another benefit of the regional approach is its improved statistical power due to reduction in the number of multiple comparisons involved relative to sensor-wise analysis.

The embodiments above are intended to be illustrative and not limiting. Additional embodiments are within the claims. In addition, although aspects of the present invention have been described with reference to particular embodiments, those skilled in the art will recognize that changes can be made in form and detail without departing from the spirit and scope of the invention, as defined by the claims.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims that are included in the documents are incorporated by reference into the claims of the present Application. The claims of any of the documents are, however, incorporated as part of the disclosure herein, unless specifically excluded. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A system for quantifying neurophysiologic activity of a subject, the system comprising:
a data input configured to receive a set of subject data representing a time series of neurophysiologic activity acquired by each of a multiplicity of spatially distributed sensors arranged to detect neural signaling in the subject; and
a data processor that includes computer hardware, the data processor being communicatively coupled to the data input and programmed to process the set of subject data to:
associate a time series of data obtained from each of the sensors with a corresponding neural population within the brain of the subject;
determine interaction sets among at least two neural populations in the brain of the subject, wherein the interaction sets are determined based on a statistical analysis of a plurality of time series of data from a corresponding plurality of sensors;
store a plurality of regional groupings of neural populations, wherein each one of the plurality of regional groupings encompasses a plurality of neural populations having a predefined relationship; and
produce an aggregated representation of cross-regional interactions between the neural populations across a selected plurality of the regional groupings based on a selected subset of the interaction sets.

2. The system of claim 1, wherein the data processor is further programmed to identify an intra regional aggregated representation of the intra-regional interactions of neural populations within that regional grouping.

3. The system of claim 1, wherein the subject data includes data generated by an instrument selected from the group consisting of: a magnetoencephalography instrument, an electroencephalography instrument, a functional magnetic resonance imaging instrument, a functional positron emission tomography instrument, or any combination thereof.

4. The system of claim 1, wherein the statistical analysis includes:
a computation of a prewhitened time series of the set of subject data, and a computation of partial cross correlations of the prewhitened time series to produce estimates of strength and sign of signaling between the groups of sensors.

5. The system of claim 1, wherein the interaction sets among at least two neural populations are interactions between pairs of neural populations.

6. The system of claim 1, wherein the interactions of the interaction sets are temporal interactions occurring within about +/−25 ms.

7. The system of claim 1, wherein the plurality of regional groupings are defined based on spatially-delineated brain regions.

8. The system of claim 1, wherein the plurality of regional groupings are defined based on functionally-delineated brain regions.

9. The system of claim 1, wherein the plurality of regional groupings are defined based on various brain structures.

10. The system of claim 1, wherein the plurality of regional groupings are defined based on the predefined relationship of a distance between groupings of sensors.

11. The system of claim 1, wherein the aggregated representation is aggregated based on at least one statistical aggregation selected from the group consisting of: average, mode, median, or any combination thereof.

12. The system of claim 1, wherein the aggregated representation is aggregated based on a temporal grouping of interactions between groups of neural populations.

13. The system of claim 1, wherein the data processor is further programmed to generate a set of global measures corresponding to a first subset of the interaction sets having a relatively short spatial distance, and a second subset of the interaction sets having a relatively long spatial distance.

14. The system of claim 1, wherein the data processor is further programmed to generate global measures based on a proportion of sensor interaction sets having correlation values significantly less than zero; a proportion of sensor interaction sets having correlation values significantly greater than zero; and a proportion of sensor interaction sets that are not significantly different from zero.

15. A method for quantifying neurophysiologic activity of a subject, using a computer system having a data processor that includes computer hardware, the method comprising:
receiving a set of subject data representing a time series of neurophysiologic activity acquired by each of a multiplicity of spatially distributed sensors arranged to detect neural signaling in the subject; and
associating a time series of data obtained from each of the sensors with a corresponding neural population within the brain of the subject;
determining interaction sets among at least two neural populations in the brain of the subject based on a statistical analysis of a plurality of time series of data from a corresponding plurality of sensors;
storing a plurality of regional groupings of neural populations, wherein each one of the plurality of regional groupings encompasses a plurality of neural populations having a predefined relationship; and
producing an aggregated representation of cross-regional interactions between the neural populations across a selected plurality of the regional groupings based on a selected subset of the interaction sets.

16. The method of claim 15, further comprising identifying an intra-regional aggregated representation of the intra-regional interactions of neural populations within that regional grouping.

17. The method of claim 15, further comprising defining the plurality of regional groupings based on at least one predefined relationship selected from the group consisting of: spatially-delineated brain regions, functionally-delineated brain regions, commonality within a brain structure.

18. The method of claim 15, further comprising defining the plurality of regional groupings based on the predefined relationship of a distance between groupings of sensors.

19. The method of claim 15, further comprising generating a set of global measures corresponding to a first subset of the interaction sets having a relatively short spatial distance, and a second subset of the interaction sets having a relatively long spatial distance.

20. The method of claim 15, further comprising generating global measures based on a proportion of sensor interaction sets having correlation values significantly less than zero; a proportion of sensor interaction sets having correlation values significantly greater than zero; and a proportion of sensor interaction sets that are not significantly different from zero.

21. A computer-readable medium comprising instructions that are adapted to cause a computer system to:
receive a set of subject data representing a time series of neurophysiologic activity acquired by each of a multiplicity of spatially distributed sensors arranged to detect neural signaling in the subject;
associate a time series of data obtained from each of the sensors with a corresponding neural population within the brain of the subject;
determine interaction sets among at least two neural populations in the brain of the subject based on a statistical analysis of a plurality of time series of data from a corresponding plurality of sensors;
store a plurality of regional groupings of neural populations, wherein each one of the plurality of regional groupings encompasses a plurality of neural populations having a predefined relationship; and
produce an aggregated representation of cross-regional interactions between the neural populations across a selected plurality of the regional groupings based on a selected subset of the interaction sets.

22. A method for quantifying neurophysiologic activity of a subject, using a computer system having a data processor that includes computer hardware, the method comprising:
transmitting a set of subject data representing a time series of neurophysiologic activity acquired by each of a multiplicity of spatially distributed sensors arranged to detect neural signaling in the subject; and
in response to the transmitting, receiving a result of processing of the set of subject data, the set of subject data having been processed such that:
a time series of data obtained from each of the sensors is associated with a corresponding neural population within the brain of the subject;
interaction sets among at least two neural populations in the brain of the subject are determined based on a statistical analysis of a plurality of time series of data from a corresponding plurality of sensors;
a plurality of regional groupings of neural populations is stored, with each one of the plurality of regional groupings encompassing a plurality of neural populations having a predefined relationship; and
an aggregated representation of cross-regional interactions between the neural populations across a selected plurality of the regional groupings is produced and transmitted based on a selected subset of the interaction sets.

23. The method of claim 22, wherein the set of subject data has further been processed such that an intra-regional aggregated representation of the intra-regional interactions of neural populations within that regional grouping is identified.

24. The method of claim 22, wherein the set of subject data has further been processed to define the plurality of regional groupings based on at least one predefined relationship selected from the group consisting of: spatially-delineated brain regions, functionally-delineated brain regions, commonality within a brain structure.

25. The method of claim 22, wherein the set of subject data has further been processed to define the plurality of regional groupings based on the predefined relationship of a distance between groupings of sensors.

26. The method of claim 22, wherein the set of subject data has further been processed to generate and transmit a set of global measures corresponding to a first subset of the interaction sets having a relatively short spatial distance, and a second subset of the interaction sets having a relatively long spatial distance; and
wherein the method further comprises receiving the set of global measures.

27. The method of claim 22, wherein the set of subject data has further been processed to generate and transmit global measures based on a proportion of sensor interaction sets having correlation values significantly less than zero; a proportion of sensor interaction sets having correlation values significantly greater than zero; and a proportion of sensor interaction sets that are not significantly different from zero; and
wherein the method further comprises receiving the global measures.
